(19) United States
(12) Plant Patent Application Publication
(10) Pub. No.: US 2013/0025011 P1
(43) Pub. Date: Jan. 24, 2013

(54) Ptilotus plant named 'B123'

(50) ;

(71) Applicants: Kok Kiong Lee, Robertson (AU); Dion Harrison, Karana Downs (AU); Margaret Johnston, Rangeville Toowoomba (AU); Daryl Clifford Joyce, Karalee (AU)

(72) Inventors: Kok Kiong Lee, Robertson (AU); Dion Harrison, Karana Downs (AU); Margaret Johnston, Rangeville Toowoomba (AU); Daryl Clifford Joyce, Karalee (AU)

(73) Assignee: The University of Queensland

(21) Appl. No.: 13/507,688

(22) Filed: Jul. 20, 2012

(30) Foreign Application Priority Data
Jul. 21, 2011 (AU) 2011/172

(51) Int. Cl. A01H 5/00

(52) U.S. Cl. PLT/263.1

(57) ABSTRACT

A new and distinct Ptilotus plant characterized by upright plant form, short plant height, very high basal branching, numerous inflorescences with up to 4 heads per primary stem, inflorescence shape that is ovoid to cylindrical, and attractive dark green leaves with hairs and undulating margins.

### CROSS-REFERENCE TO RELATED APPLICATION

This application claims priority to Australian Plant Breeder's Rights Application No. 2011/172 filed on Jul. 21, 2011.

### BOTANICAL DENOMINATION

Ptilotus hybrid (P. nobilis×P. exaltatus var. semilanatus)

### VARIETY DESIGNATION

‘B123’

### BACKGROUND

The present invention relates to a new and distinct cultivar of Ptilotus and given the cultivar name of ‘B123’. The new variety arose from a series of scientific experiments performed to investigate the breeding compatibility of Ptilotus nobilis and Ptilotus exaltatus, with the intention of producing an interspecific hybrid. The new variety was derived via controlled pollination in an insect-proof glasshouse from crosses involving 18 fully developed florets on the one inflorescence of maternal parent P. nobilis Pn1 (Cunnamulla) (unpatented), which were emasculated prior to anther dehiscence on the day of anthesis. Maternal florets were hand pollinated using pollen from paternal parent P. exaltatus var. semilanatus Pes2 (unpatented). During the experiment, both maternal and paternal parents' inflorescences were bagged to prevent contamination with unwanted pollen. Only one viable seed set from this cross combination which was sown in tissue culture on deFossards basal medium. The germinated seedling was deflasked and grown in the nursery.

The new variety was noted to be very attractive with its multiple pink inflorescences on a short upright plant (30 cm high) with dark green foliage, and was selected for further evaluation noting its numerous inflorescences (total 31 visible inflorescences on 10 primary stems), and pink flower color. The new variety was chosen having the following unique combination of characteristics: upright plant form, short plant height, very high basal branching, numerous inflorescences with up to 4 heads per primary stem, inflorescence ovoid to cylindrical in shape, and attractive dark green leaves with hairs and undulating margins.

Compared to parent Pn1, ‘B123’ has a light to mid pink-purple inflorescence instead of cream-green, has a capitate to sub-conical inflorescence shape instead of cylindrical to conical, and has leave hairs while in Pn1 they are absent. Compared to parent Pes2, ‘B123’ has a light to mid pink-purple inflorescence instead of dark purple, has a capitate to sub-conical inflorescence shape instead of semi-globose, and an erect plant growth habit instead of spreading.

A field trial undertaken at Redlands Bay, QLD, Australia between November 2008 and March 2009 revealed superior garden performance of the new variety compared to the other Ptilotus cultivars in the trial (P. nobilis cv. ‘Passion’, P. nobilis cv. ‘Poise’ and P. nobilis cv. ‘Purity’, all unpatented). Final selection of the new variety was based on its consistently high propagation efficiency from cuttings as determined from a series semi-commercial production trials conducted between February 2009 and August 2010.

This new cultivar has been reproduced only by asexual propagation (cuttings). Each of the progeny exhibits identical characteristics to the original plant. Asexual propagation by cuttings using standard micropropagation techniques, as done in Galton, QLD, Australia, shows that the foregoing characteristics and distinctions come true to form and are established and transmitted through succeeding propagations. The present invention has not been evaluated under all possible environmental conditions. The phenotype may change with variations in environment without a change in the genotype of the plant.

### BRIEF DESCRIPTION OF THE DRAWINGS

FIG. 1 shows an inflorescence from ‘B123’ and from its paternal parent.

FIG. 2 shows an inflorescence from ‘B123’ and from ‘Passion’ (unpatented).

FIG. 3 shows an inflorescence from ‘B123’ and from ‘Joey’ (unpatented).

FIG. 4 shows ‘B123’ and ‘Passion’ plants growing in a 140 mm pot.

FIG. 5 shows the flowering stem from ‘B123’ and ‘Passion’. ‘B123’ has up to 4 inflorescences per primary stem, while ‘Passion’ usually has 1, but can have up to 2 inflorescences per primary stem.

FIGS. 6 and 7 show the leaves from ‘B123’ and ‘Passion’.

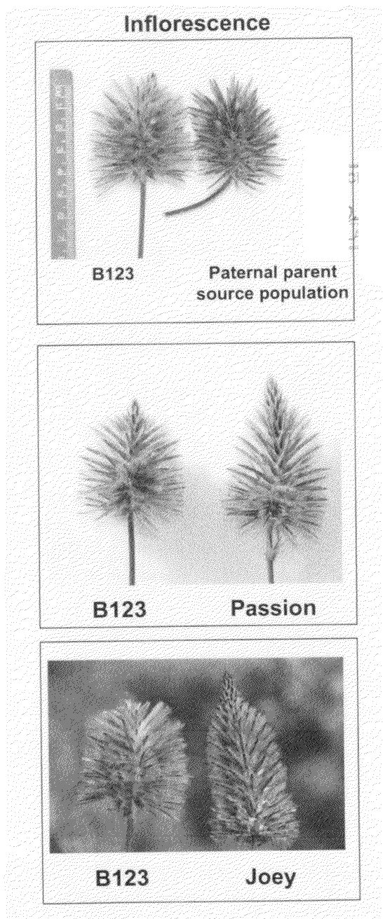

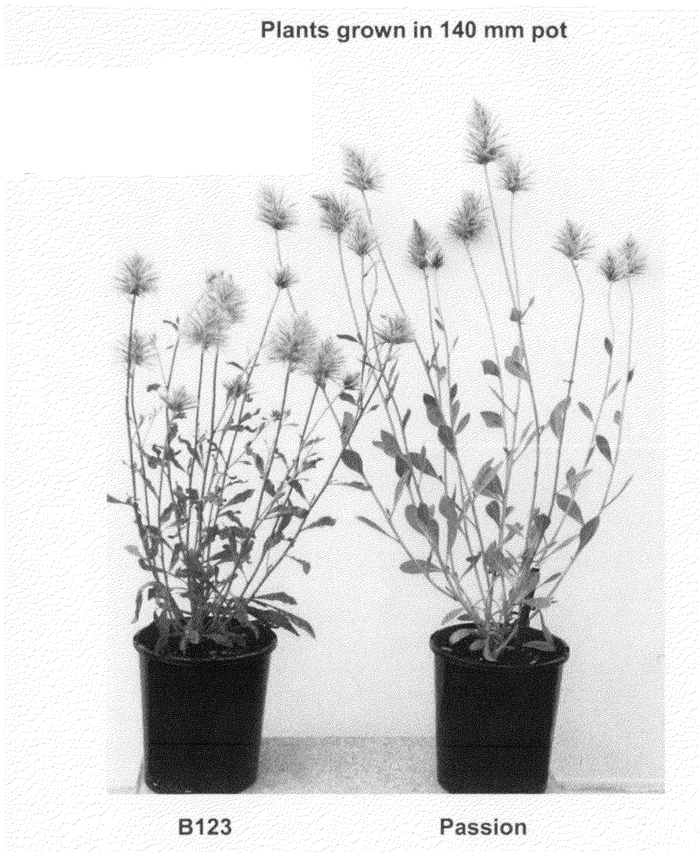

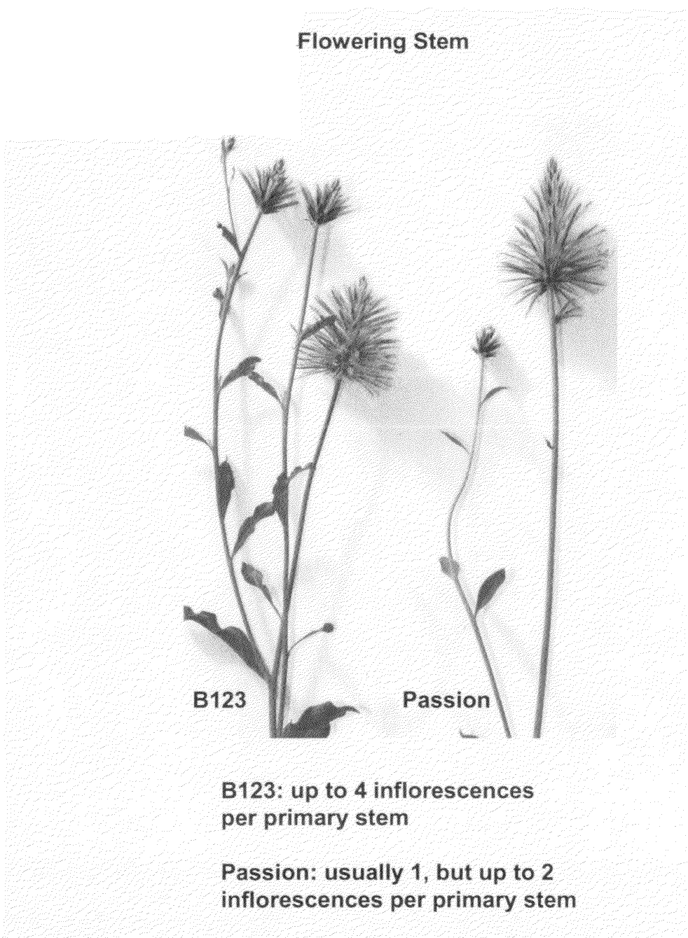

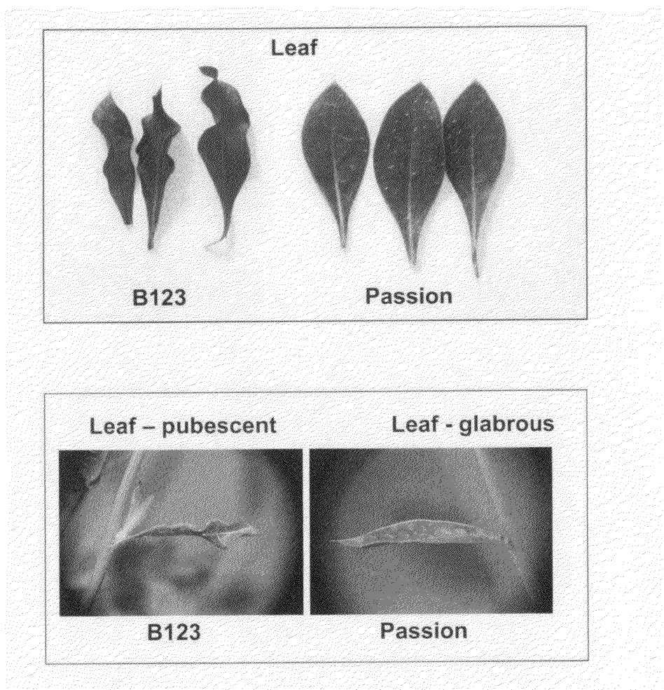

### DETAILED PLANT DESCRIPTION

The following is a detailed description of the new Ptilotus cultivar based on observations taken in October 2011 through February 2012 of plants propagated by cuttings and grown in Gatton, QLD, Australia in 140 mm pots in a soil-less medium under outdoor conditions, fertilized with controlled release fertilizer and drip irrigated. Measurements were taken from 20 plants per variety. The color descriptions are all based on The Royal Horticultural Society Colour Chart, 2007 edition.
Plant: Non woody
   Height.—about 35.3 cm tall.
   Density.—dense.
Leaf:
   Variegation.—absent.
   Color of upper side.—137A.
   Shape.—pubescent.
   Hair.—present.
   Margin.—strong undulation.
   Glossiness of upper side.—medium.
Inflorescence:
   Size.—about 4.28 cm wide and 5.33 cm long.
   Number per plant.—about 26.
   Number per primary branch.—4.
   Shape.—ovoid to cylindrical.
   Tepal blade venation color.—198D.
   Tip shape.—mildly mucronate.
Flower:
   Type.—single.
   Color.—N74A.
Pest and disease tolerance: The new cultivar is typical to the genus. No known resistances to pests or diseases. No problems have been found in Gatton, QLD, Australia.

Table 1 shows a comparison of known Ptilotus varieties to the new ‘B123’ variety.

TABLE 1
Comparison of other Ptilotus varieties to the new ‘B123’ variety.

| | | | | Expression in | |
|---|---|---|---|---|---|
| | Distinguishing | | Expression in | Comparator | |
| Variety | Characteristics | | ‘B123’ | Variety | Comments |
| ‘Pes2’ | Inflorescence | overall | light to mid pink- | dark purple | P. exaltatus |
| (unpatented) | | coloration | purple | | var. |
| | | | | | semilanatus; |
| | | | | | parent line |
| ‘Poise’ | Inflorescence | overall | light to mid pink- | cream tan/pink | |
| (unpatented) | | coloration | purple | | |
| ‘Pn1’ | Inflorescence | overall | light to mid pink- | cream-green | P. nobilis; |
| (unpatented) | | coloration | purple | | parent line |
| ‘Purity’ | inflorescence | overall | light to mid pink- | cream-green | |
| (unpatented) | | coloration | purple | | |
| ‘Joey’ | Inflorescence | overall | light to mid pink- | bright pink | P. exaltatus |
| (unpatented) | | coloration | purple | | |
| ‘Musk | Inflorescence | overall | light to mid pink- | bright pink | P. exaltatus |
| Sticks’ | | coloration | purple | | |
| (unpatented) | | | | | |
| ‘Platinum | Inflorescence | overall | light to mid pink- | silvery bright pink- | P. exaltatus |
| Wallaby’ | | coloration | purple | purple | |
| (U.S. Plant | | | | | |
| Pat. No. | | | | | |
| PP20,785) | | | | | |

The most similar variety to ‘B123’ is ‘Passion’ (Unpatented). A comparison is provided in Table 2.

TABLE 2
Comparison of traits which distinguish ‘B123’ from ‘Passion’

| Characteristics | ‘B123’ | ‘Passion’ |
|---|---|---|
| plant density | dense | sparse to medium |
| Plant height | Short | medium |
| Stem: degree of hairiness | Medium | Very low |
| Leaf: width of blade | very narrow | narrow |
| Leaf: presence of hairs | present | absent |
| Leaf: undulation of the margin | strong | absent or very weak |
| Leaf: shape of cross-section | concave | flat |
| Leaf: glossiness of upper side | medium | very weak to weak |
| Leaf: primary color | 137A | 138A |
| Inflorescence: maximum # of | 4 | 2 |
| heads per primary branch | | |
| Inflorescence: shape | ovoid to cylindrical | cylindrical to conical |
| Inflorescence: tepal blade | 198D | 201C |
| ventaion color | | |
| Inflorescence: tip shape | mildly mucronate | acute |
| Inflorescence: width (cm) | | |
| Mean | 4.28 | 4.58 |
| Std. Deviation | 0.10 | 0.09 |
| LSD/sig | 0.08 | P ≦ 0.01 |
| Inflorescence number per plant | | |
| Mean | 25.95 | 18.05 |
| Std. Deviation | 5.19 | 4.58 |
| LSD/sig | 4.19 | P ≦ 0.01 |
| Plant: height (cm) | | |
| Mean | 35.30 | 44.87 |
| Std. Deviation | 3.40 | 4.21 |
| LSD/sig | 3.3 | P ≦ 0.01 |
| Inflorescence: length (cm) | | |
| Mean | 5.33 | 9.08 |
| Std. Deviation | 0.79 | 1.28 |
| LSD/sig | 0.91 | P ≦ 0.01 |

We claim:

1. A new and distinct Ptilotus plant as herein illustrated and described.
